(19) United States
(12) Patent Application Publication
(10) Pub. No.: US 2017/0049727 A1
(43) Pub. Date: Feb. 23, 2017

(54) Use of N-Acetylcysteine Amide in the Treatment of Acquired Immune Deficiency Syndrome and HIV Infection

(71) Applicant: Glenn A. Goldstein, New York, NY (US)

(72) Inventor: Glenn A. Goldstein, New York, NY (US)

(21) Appl. No.: 15/118,752

(22) PCT Filed: Feb. 12, 2015
(86) PCT No.: PCT/US2015/015543

§ 371 Date: Aug. 12, 2016

Related U.S. Application Data
(60) Provisional application No. 61/938,967, filed on Feb. 12, 2014.

(51) Int. Cl. A61K 31/16

(52) U.S. Cl. CPC A61K 31/16

(57) ABSTRACT

This disclosure describes methods of use for N-acetylcysteine amide for the treatment of HIV infection and AIDS.

### BACKGROUND

More than 60 million people have been infected with the human immunodeficiency virus (“HIV”), the causative agent of acquired immune deficiency syndrome (“AIDS”), since the early 1980s. HIV/AIDS is now the leading cause of death in sub-Saharan Africa, and is the fourth biggest killer worldwide. At the end of 2001, an estimated 40 million people were living with HIV globally.

Modem anti-HIV drugs target different stages of the HIV life cycle and a variety of enzymes essential for HIV's replication and/or survival. Nonetheless, in the vast majority of subjects none of these antiviral drugs, either alone or in combination, proves effective either to prevent eventual progression of chronic HIV infection to AIDS or to treat acute AIDS. This phenomenon is due, in part, to the high mutation rate of HIV and the rapid emergence of mutant HIV strains that are resistant to antiviral therapeutics upon administration of such drugs to infected individuals. There is a need in the art for other compounds and therapeutic aspects to treat HIV and AIDS.

### SUMMARY

The disclosure provides a method of reducing human immunodeficiency virus (HIV) integration in a mammalian cell comprising administering to the cell a compound comprising an effective amount of NACA. In certain environments, the viral integration of HIV is decreased by at least 50%. In other embodiments, the viral integration of HIV is decreased by at least 10, 20, 30, 40, 50, 60, 70, 80 or 90%. In yet other embodiments, the mammalian cells are human cells.

### BRIEF DESCRIPTION OF THE DRAWINGS

FIG. 1 is a line graph showing amount of reverse transcriptase (RT) activity in non-activated HIV infected U1 cells with varying amounts of NACA,

FIG. 2 is a bar graph showing thymidine incorporation in HIV U1 cells in media (RPMI), and activated with TNF-α or IL-6 with varying amounts of NACA.

FIG. 3 shows bar graphs showing reverse transcriptase (RT) activity in UI cells infected with HIV and activated with TNF-α, or IL-6 with varying amounts of NACA.

FIG. 4 is a bar graph showing vaibility of HIV infected peripheral blood mononuclear cells (PBMCs) in the presence of varying amounts of NACA.

FIG. 5 is a bar graph showing amount of HIV RNA produced in HIV infected peripheral blood mononuclear cells (PBMCs) in the presence of varying amounts of NACA.

FIG. 6 is a bar graph that shows percent inhibition of HIV replication in HIV infected peripheral blood mononuclear cells (PBMCs) in the presence of varying amounts of NACA.

FIG. 7 is a bar graph that shows reverse transcriptase (RT) activity in HIV infected U1 cells activated with TNF-α with varying amounts of NACA.

FIG. 8 is a bar graph that shows reverse transcriptase (RT) activity in HIV infected U1 cells activated with IL-6 with varying amounts of NACA.

FIG. 9 is a bar graph that shows reverse transcriptase (RT) activity in HIV infected U1 cells activated with PMA with varying amounts of NACA.

FIG. 10 is a schematic that shows a potential mechanism of NACA in inhibition of HIV replication.

FIG. 11 is a schematic that shows a potential mechanism of NACA in inhibition of HIV replication.

FIG. 12 is a schematic that shows a potential mechanism of NACA in inhibition of HIV replication.

FIG. 13A is a bar graph showing reverse transcriptase activity in cells treated with NACA and controls.

FIG. 13B is a bar graph showing protease activity in the presence of NACA and controls.

FIG. 14 is a bar graph showing integration activity in the presence of NACA and controls.

FIG. 15A is a bar graph showing the amount of p24 in the presence of NACA and controls.

FIG. 15B is a bar graph showing infectivity of HIV in the presence of NACA and controls.

### DETAILED DESCRIPTION

The present invention provides the use of N-acetylcysteine amide (NAC amide or NACA) or derivatives thereof, or a physiologically acceptable derivative, salt, or ester thereof, to treat HIV infection and diseases that result from, or are associated with HIV infection including AIDS. NACA and its derivatives are provided for use in methods and. compositions for improving and treating such disorders, conditions, pathologies and diseases.

As used herein, a “subject” within the context of the present invention encompasses, without limitation, mammals, e.g., humans, domestic animals and livestock including cats, dogs, cattle and horses. A “subject in need thereof” is a subject having one or more manifestations of disorders, conditions, pathologies, and diseases as disclosed herein in which administration or introduction of NAC amide or its derivatives would be considered beneficial by those of ordinary skill in the art.

“Therapeutic treatment” or “therapeutic effect” means any improvement in the condition of a subject treated by the methods of the present invention, including obtaining a preventative or prophylactic effect, or any alleviation of the severity of signs or symptoms of a disorder, condition, pathology, or disease or its sequelae, including those caused by other treatment methods (e.g., chemotherapy and radiation therapy), which can be detected by means of physical examination, laboratory, or instrumental methods and considered statistically and/or clinically significant by those skilled in the art.

“Prophylactic treatment” or “prophylactic effect” means prevention of any worsening in the condition of a subject treated by the methods of the present invention, as well as prevention of any exacerbation of the severity of signs and symptoms of a disorder, condition, pathology, or disease or its sequelae, including those caused by other treatment methods (e.g., chemotherapy and radiation therapy), which can be detected by means of physical examination, laboratory, or instrumental methods and considered statistically and/or clinically significant by those skilled in the art.

Another aspect of the present invention provides a compound of the formula I:

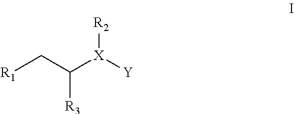

wherein: R₁ is OH, SH, or S—S-Z; X is C or N; Y is NH₂, OH, CH₃—C═O, or NH—CH₃; R.sub.2 is absent, H, or ═O R₃ is absent or

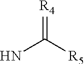

wherein: R₄ is NH or O; R₅ is CF₃, NH₂, or CH₃

and wherein: Z is

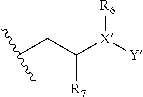

with the proviso that if R₁ is S—S-Z, X and X′ are the same, Y and Y′ are the same, R₂ and R₆ are the same, and R₃ and R₇ are the same.

The present invention also provides a NAC amide compound and NAC amide derivatives comprising the compounds disclosed herein. Other derivatives are disclosed in U.S. Pat. No. 8,354,449, incorporated by reference in its entirety.

In another aspect, a process for preparing an L- or D-isomer of the compounds of the present invention are provided, comprising adding a base to L- or D-cystine diamide dihydrochloride to produce a first mixture, and subsequently heating the first mixture under vacuum; adding a methanolic solution to the heated first mixture; acidifying the mixture with alcoholic hydrogen chloride to obtain a. first residue; dissolving the first residue in a first solution comprising methanol saturated with ammonia; adding a second solution to the dissolved first residue to produce a second mixture; precipitating and washing the second mixture; filtering and drying the second mixture to obtain a second residue; mixing the second residue with liquid ammonia and an ethanolic solution of ammonium chloride to produce a third mixture; and filtering and drying the third mixture, thereby preparing the L- or D-isomer compound.

In some embodiments, the process further comprises dissolving the L- or D-isomer compound in ether; adding to the dissolved L- or D-isomer compound an ethereal solution of lithium aluminum hydride, ethyl acetate, and water to produce a fourth mixture; and filtering and drying the fourth mixture, thereby preparing the L- or D-isomer compound.

Another aspect of the invention provides a process for preparing an L- or D-isomer of the compounds disclosed herein, comprising mixing S-benzyl-L- or D-cysteine methyl ester hydrochloride or O-benzyl-L- or D-serine methyl ester hydrochloride with a base to produce a first mixture; adding ether to the first mixture; filtering and concentrating the first mixture; repeating steps (c) and (d), to obtain a first residue; adding ethyl acetate and a first solution to the first residue to produce a second mixture; filtering and drying the second mixture to produce a second residue; mixing the second residue with liquid ammonia, sodium metal, and an ethanolic solution of ammonium chloride to produce a third mixture; and filtering and drying the third mixture, thereby preparing the L- or D-isomer compound.

HIV

In certain embodiments, NACA and derivatives thereof are used for the treatment of human immunodeficiency virus (HIV) infection and symptoms associated with acquired immune deficiency syndrome (AIDS). In some embodiments, NACA and derivatives thereof are used to reduce HIV replication in infected cells. These cells can include peripheral blood mononuclear cells (PBMCs). PDMCs include lymphocytes, monocytes and macrophages. Lymphocytes include B-cells and T-cells. T-cells include CD4 and CD8 positive T-cells. NACA and derivatives thereof can be used to reduce HIV replication in any of these cell types. In certain embodiments, replication is reduced, greater than 10, 15, 20, 25, 30, 35, 40, 45, 50, 55, 60, 65, 70, 75, 80, 85, 90, 95, 96, 97, 98, 99 or 100%.

In certain embodiments, NACA and derivatives thereof can be used to reduce reverse transcriptase (RT) activity in HIV infected cells. These cells can include peripheral blood mononuclear cells (PBMCs). PDMCs include lymphocytes, monocytes and macrophages. Lymphocytes include B-cells and T-cells. T-cells include CD4 and CD8 positive T-cells. NACA and derivatives thereof can be used to reduce RT activity in any of these cell types. In certain embodiments, RT activity is reduced, greater than 10, 15, 20, 25, 30, 35, 40, 45, 50, 55, 60, 65, 70, 75, 80, 85, 90, 95, 96, 97, 98, 99 or 100%.

According to certain embodiments, NACA or derivatives thereof can be administered systemically. Systemic administration methods include intraperitoneal, intravenous or oral administration. Doses, amounts or quantities of NACA, or derivatives thereof, are determined on an individual basis. As is appreciated by the skilled practitioner in the art, dosing is dependent on the severity and responsiveness of the cataract to be treated, but will normally be one or more doses per day, with course of treatment lasting from several days to several months, or until a cure is effected or a diminution of disease state is achieved. Persons ordinarily skilled in the art can easily determine optimum dosages, dosing methodologies and repetition rates. For example, a pharmaceutical formulation for orally administrable dosage form can comprise NACA, or a pharmaceutically acceptable salt, ester, or derivative thereof in an amount equivalent to at least 25-500 mg per dose, or in an amount equivalent to at least 50-350 mg per dose, or in an amount equivalent to at least 50-150 mg per dose, or in an amount equivalent to at least 25-250 mg per dose, or in an amount equivalent to at least 50 mg per dose.

In some embodiments, administration of NACA or derivatives thereof to reduce symptoms associated with HIV infection in a subject in need thereof. Administration of therapeutically effective amount of NACA or a derivative thereof can improve symptoms associated with AIDS. These symptoms include various immune dysfunctions that can present as fever, fatigue, swollen lymph nodes, diarrhea, weight loss, cough, shortness of breath, night sweats, chills and skin pathology. In other embodiments, NACA or derivatives thereof can be co-administered with any known HIV or AIDS therapeutic. These therapeutics include fusion inhibitors, CCR5 receptor antagonists, nucleoside reverse transcriptase inhibitors, non-nucleoside reverse transcriptase inhibitors, protease inhibitors, integrase inhibitors and maturation inhibitors.

Pharmaceutical Compositions

As used herein the term “pharmaceutical composition” refers to a preparation of one or more of the components described herein, or physiologically acceptable salts or prodrugs thereof, with other chemical components such as physiologically suitable carriers and excipients. The purpose of a pharmaceutical composition is to facilitate administration of a compound to an organism. The term “prodrug” refers a precursor compound that can hydrolyze, oxidize, or otherwise react under biological conditions (in vitro or in vivo) to provide the active compound. Examples of prodrugs include, but are not limited to, metabolites of NSAIDs that include biohydrolyzable moieties such as biohydrolyzable ainides, biohydrolyzable esters, biohydrolyzable carbarnates, biohydrolyzable carbonates, biohydrolyzable ureides, and biohydrolyzable phosphate analogues.

The term “excipient” refers to an inert or inactive substance added to a pharmaceutical composition to further facilitate administration of a compound. Non-limiting examples of excipients include calcium carbonate, calcium phosphate, various sugars and types of starch, cellulose derivatives, gelatin, vegetable oils and polyethylene glycols.

The pharmaceutical compositions of the present invention comprise NAC Amide or derviate thereof and may also include one or more additive drug (e.g., additional active ingredients), such as, but not limited to, NSAIDs, antibiotics, conventional anti-cancer and/or anti-inflammatory agents that may be suitable for combination therapy.

The pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

The term “administration” or any lingual variation thereof as used herein is meant any way of administration. The one or more of NAC Amide or derivative thereof and at least one additional drug may be administered in one therapeutic dosage form or in two separate therapeutic dosages such as in separate capsules, tablets or injections. In the case of the two separate therapeutic dosages, the administration may be such that the periods between the administrations vary or are determined by the practitioner. It is however preferred that the second drug is administered within the therapeutic response time of the first drug. The one or more of NAC Amide or derivative thereof and at least one additional drug which may be administered either at the same time, or separately, or sequentially, according to the invention, do not represent a mere aggregate of known agents, but a new combination with the valuable property that the effectiveness of the treatment is achieved at a much lower dosage of said at least one additional drug.

The pharmaceutical compositions of the present invention may be administered by any convenient route, for example, by infusion or bolus injection, by absorption through epithelial or mucocutaneous (e.g., oral mucosa, rectal and intestinal mucosa, etc.)and may be administered together with any other therapeutic agent. Administration can be systemic or local.

Various delivery systems are known, e.g., encapsulation in liposomes, microparticles, microcapsules or capsules, that may be used to administer the compositions of the invention. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically to the cars, nose, eyes, or skin. The preferred mode of administration is left to the discretion of the practitioner, and will depend in part upon the site of the medical condition (such as the site of cancer) and the severity of thereof.

For example, for injection the composition of the invention may be formulated in aqueous solutions, preferably in physiologically compatible. buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the composition can be formulated readily by combining the active components with any pharmaceutically acceptable carriers known in the art. Such “carriers” may facilitate the manufacture of such as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

Pharmaceutical compositions, which car be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Dyestuffs or pigments may be added to the tablets or dragee coatings for identification or to characterize different combinations of active NSAID doses. In addition, stabilizers may be added.

Pharmaceutical compositions for parenteral administration include aqueous solutions of the active preparation in a water-soluble form. Additionally, suspensions of the active preparation may be prepared as oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl, cellulose, sorbitol or dextran. Optionally, the suspension may also contain suitable stabilizers or agents, which increase the solubility of the compounds, to allow for the preparation of highly concentrated solutions.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

### EXAMPLES

### Example 1

### Treatment of HIV with NACA

In some embodiments, the present invention relates to a method for inhibiting HIV replication. Data is attached reporting the results of an HIV study in lymphocytes with 100% block of replication at 20 mM concentration.

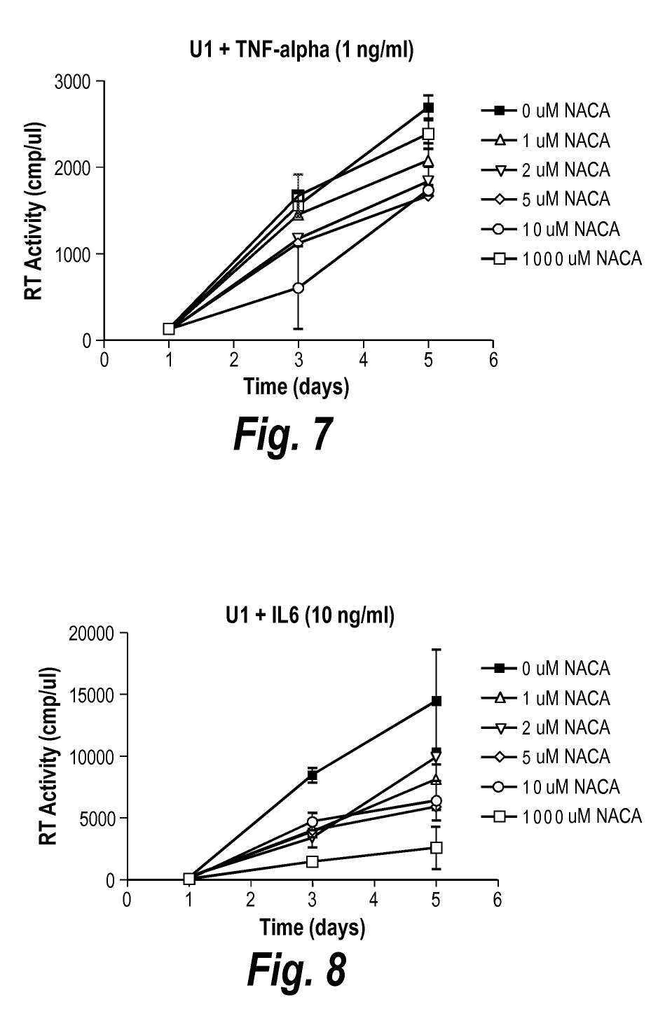

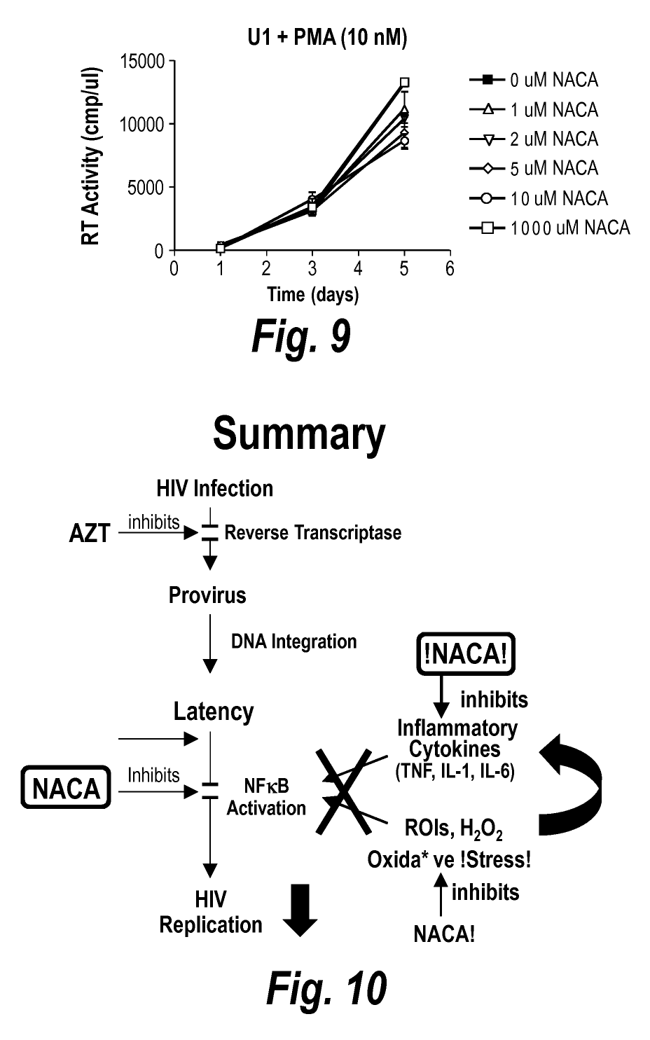

Chronically HIV-infected U1 cells (monocytes) were stimulated for 6 hours in the presence of NACA. Cytokines (IL-6, TNF-a) or PMA were added and maintained in the cell cultures for 6 days, collecting supernatants daily. Cell viability and proliferation were checked by optical microscopy, and [3H]-thymidine incorporation, and calcein AM assay. Reverse transcriptase-polymerase chain reaction (RT-PCR) and reverse transcriptase (RT) activity assay as readout of virion production. As shown in FIG. 7, addition of NACA did not alter relative amounts of reverse transcriptase activity between days 3 and 5. FIG. 8 shows that addition of NACA did not have a large effect on U1 cell viability or proliferation as measured by [3H]-thymidine incorporation, 10-333 μM NACA is effective in reducing HIV replication in activated U1 cells. FIG. 9 shows significant reduction to reverse transcriptase activity in U1 cells activated with 1 ng/mL TNF-α or 10 ng/mL IL-6.

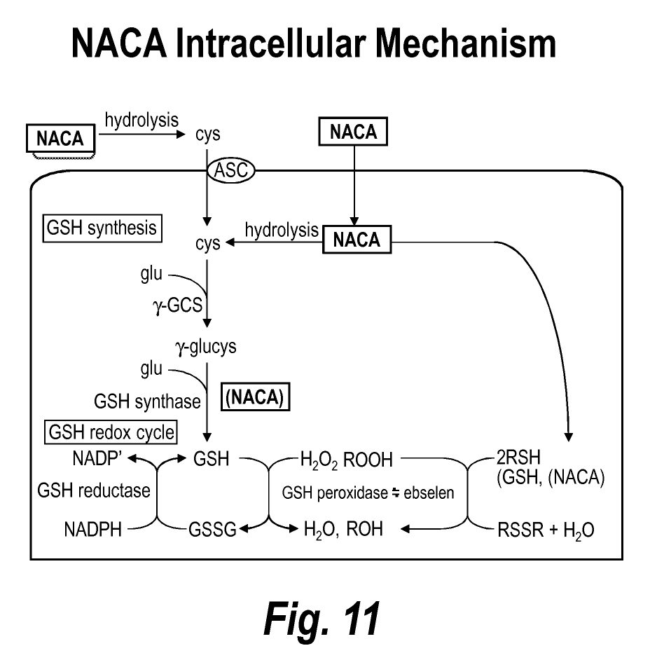

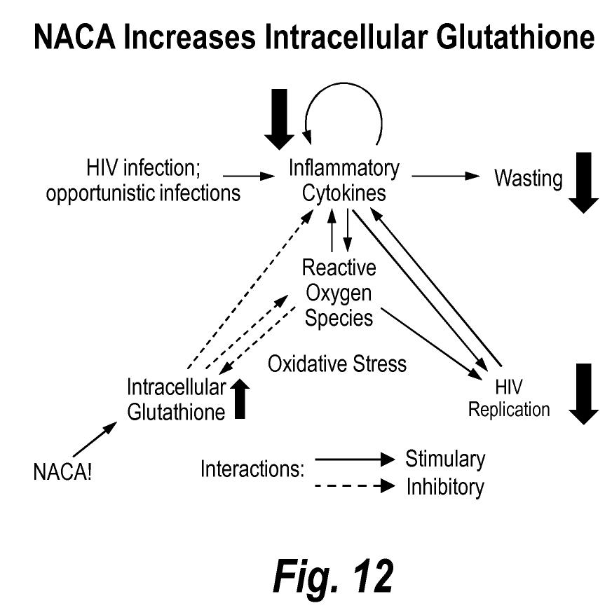

Peripheral blood mononuclear cells (PBMC) were acutely infected with HIV-1 and stimulated with NACA. Cell viability was measured in HIV infected PBMCs with varying concentrations of NACA. FIG. 10 shows that administration of NACA did not affect cell viability. However, NACA had an inhibitor effect on HIV replication in HIV infected PBMCs as shown in FIG. 11. At concentrations of 10 mM and above, HIV replication was almost 100% inhibited as shown in FIG. 12.

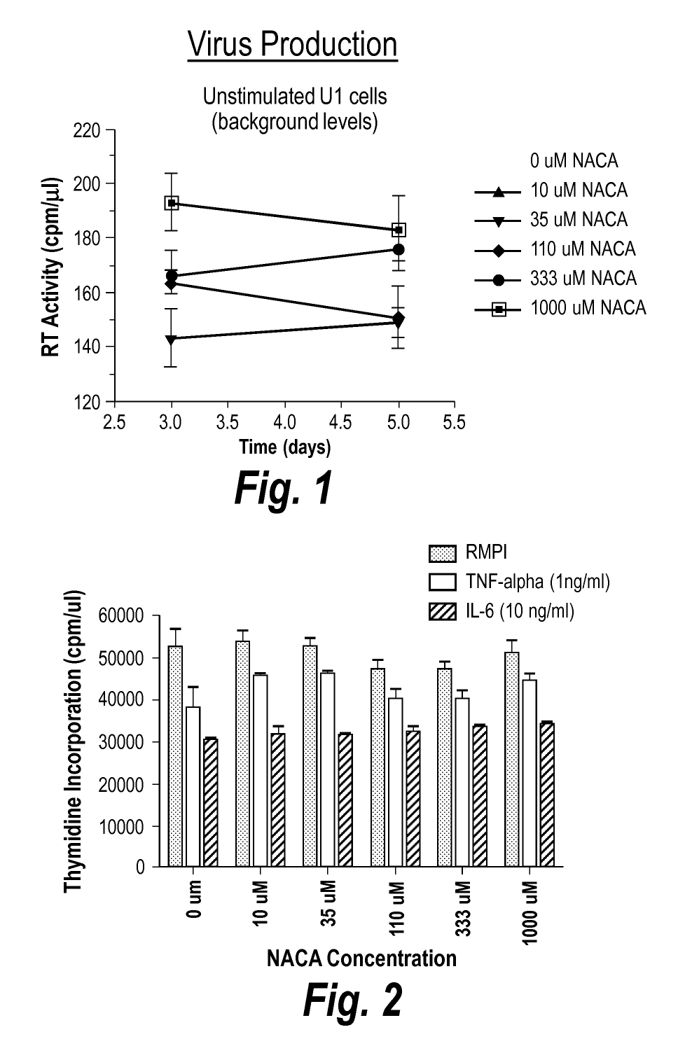

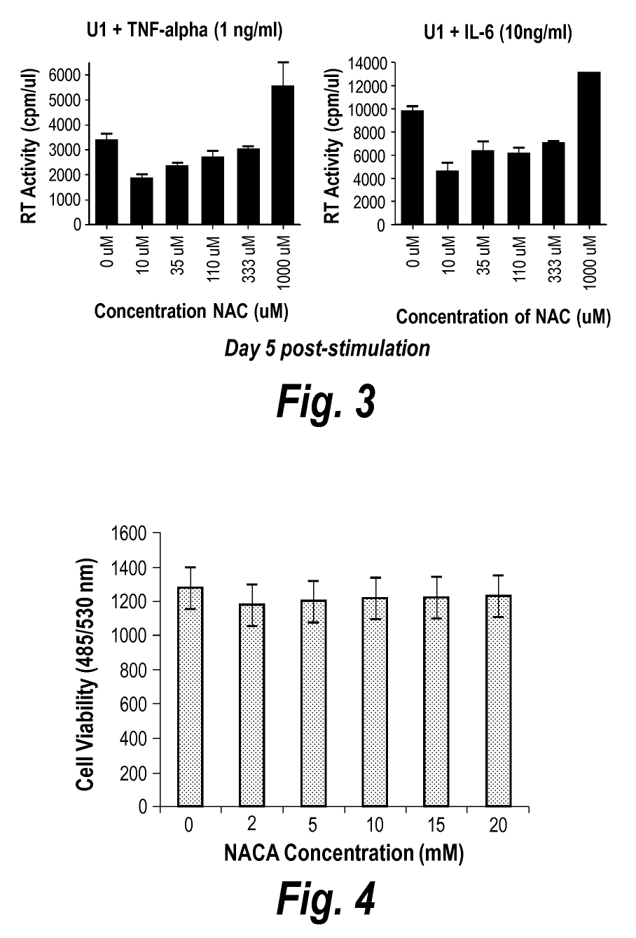

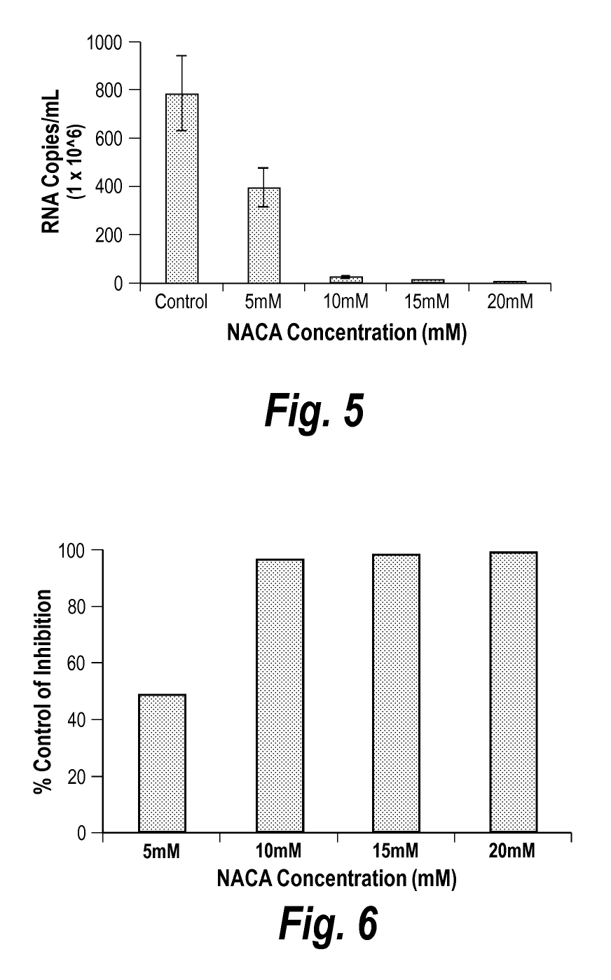

RT activity was also measured in activated U1 cells. U1 cells were activated with 1 ng/mL TNF-α, 10 ng/mL IL-6 or 10 nM phorbol 12-myristate 13-acetate (PMA). As shown in FIGS. 13-15, 1-10 μM NACA is effective in reducing HIV replication in activated U1 cells.

Applicants do not wish to be limited by theory, however it is possible that NACA inhibits HIV replication by preventing NFKB activation both by reducing reactive oxygen species and the effect of inflammatory cytokines as shown in FIG. 16. NACA may also act by increasing intracellular glutathione as shown in FIGS. 17 and 18.

### Example 2

1. Materials and Methods

1.1. Materials

The cell lines ACH-2 (Clouse et al 1989; Terwilliger et al 1988) and TZM-b1 (Wei et al 2002) were obtained from the NIH AIDS Research & Reference Reagent Program (Germantown, Md., USA). Human T-lymphocytic CEM cells were obtained from the American Type Culture Collection (Rockville, Md., USA). Recombinant HIV-1 protease was purchased from AnaSpec Inc. (Fremont, Calif., USA) and 12-phorbol-13-myristate acetate (PMA) from Sigma-Aldrich (Stockholm, Sweden).

1.2. Inhibition of HIV-1 Replication and Cytostatic Activity Assay

CEM cells were suspended in fresh culture medium and infected with HIV-1 (IIIB) or HIV-2(ROD) at 100 CCID50 per ml of cell suspension. 100 μl of the infected cell suspension were transferred to microplate wells, mixed with 100 μl of the appropriate dilutions of compounds, and further incubated at 37° C. After 4 days, giant cell formation was recorded microscopically and the number of giant cells was estimated as percentage of the number of giant cells present in the non-treated virus-infected cell cultures.

The cytostatic activity assay was performed in 96-well microtiter plates. 5-7.5×10⁴ cells and a given amount of the test compound were added to each well. The cells were allowed to proliferate for 72 h at 37° C. in a humidified CO₂-controlled atmosphere. At the end of the incubation period, the cells were counted in a Coulter counter. The IC₅₀ (half maximal inhibitory concentration) was defined as the concentration of the compound that inhibited cell proliferation by 50%.

1.3. Viability Test

CEM cells (3×10⁵ cells/m1) were seeded and grown in the presence or absence of indicated concentrations of NACA. The medium was replaced and added new NACA after two days. After 48 and 96 hours, respectively, aliquots of the cells were harvested and combined with trypan blue dye before analyzed. microscopically on a hemocytometer counting chamber. Blue cells were counted as nonviable and to estimate the amount of viable cells the amount of live cells was compared to the total amount of cells.

1.4. Virus Expression

ACH-2 cells (1×10⁶ cells/nil) were cultured with 100 nM PMA. After three days the cell culture supernatant was collected, cleared by centrifugation at 300×g for 10 min and passed through a 0.45 μm filter. p24 content was measured in an Architect i2000SR (Abbott Laboratories, Illinois, USA) as a measurement for virus content and supernatant was either used directly or frozen at −80° C. for later usage.

1.5. Reverse transcriptase (RT) Inhibition

The ability of the compounds to inhibit the activity of viral RT was determined by a Lenti RT Activity kit (Cavidi, Uppsala, Sweden) according to the manufacturer's instructions (protocol A): A poly-A plate was incubated with the reaction buffer for up to 1 h at 33° C. Afterwards, the viral sample together with NACA, an inhibition control (nevirapin) or a vehicle control (DMSO) were added and incubated overnight at 33° C. The plate was washed, exposed to the tracer for 90 min at 33° C. and washed again, before the substrate was added. The plate was incubated in the dark and subsequently read at 405 nm in a Multiskan RC (Labsystem).

1.6. Protease Inhibition

The ability of NACA to inhibit the activity of recombinant HIV-1 protease was determined by a SensoLyte 520 HIV-1 protease assay kit (AnaSpec Inc.) according to manufacturer's instructions: The compounds and the HIV-1 protease were added to a dark-bottom 96 well plate and the following controls were included: positive control (without NACA), inhibition control (pepstatin A), vehicle control (DMSO), NACA controls (to test for autofluorescence)and substrate control (assay buffer only). The plate as well as the HIV-1 protease substrate solution were incubated 15 minutes at 37° C. and 50 μl protease substrate was added to all wells. The fluorescence was measured at Ex/Em=490 nm/520 nm with a Varioskan Flash (Thermo Electron Corporation

1.7. Infectivity Assay

1×10⁵ TZM-b1 cells were infected with diluted virus suspension (see 2.3 Virus expression) in a total of 0.2 ml DMEM medium in the presence of NACA, an inhibition control (nevirapin) or a vehicle control (DMSO). After 40 h at 37° C., the cells were assayed in a Luminoskan Ascent (Thermo Labsystems) for luciferase activity using the OneGlo luciferase assay system (Promega).

To measure the infectivity of particles produced in the presence of the compounds, ACH-2 cells (1×10⁶ cells/ml) were cultured with 100 nM PMA in the presence of the indicated amount of NAGA or DMSO (negative control). After 3 days, the p24 content of the clarified supernatant(see 2.3 Virus expression) was measured. The supernatant was used for an infectivity test (see 2.4 Infectivity test) without the addition of any compound to the wells. The final dilution of the viral supernatant in the infectivity test was 1:100. A control experiment was performed, in which virus particles produced without the addition of NACA, were used for the infectivity test.

1.8. Statistical Analysis

Statistical analysis was performed with the two-tailed, paired Student's t-test. P-values are indicated if significant (*: p <0.05; **: p <0.01; ***: p <0.001).

2. Results

2.1. Inhibition of Viral Replication

NACA was evaluated for its inhibitory activity against HIV-1 and HIV-2 replication by quantification of virus-induced syncytia formation as well as its cell growth inhibitory properties in cell culture. NACA showed an activity against both used strains (HIV-1(IIIB) and HIV-2 (ROD)) at concentrations around 230 μM (Table 1), while the IC₅₀ value for CEM cells was approx. 564 μM. However, an additional experiment showed that the CEM cells were alive (more than 99%) at concentrations up to 10 mM and only inhibited in their proliferation (data not shown).

TABLE 1
Inhibition of viral replication

| | IC₅₀ | | | Ratio IC₅₀/ | |
|---|---|---|---|---|---|
| | (μM) * | EC₅₀ (μM) ^{#} | | EC₅₀ (CEM) | |
| Compound | CEM | HIV-1 | HIV-2 | HIV-1 | HIV-2 |
| NACA | 564 ± 98 | 224 ± 86 | 234 ± 0 | 3 | 2 |
| 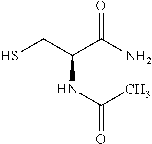 | (CC₅₀) | | | | |

* 50% inhibitory concentration
^{#} effective concentration or concentration required to protect CEM cells against the cytopathogenicity of HIV by 50%

2.2. NACA Does Not Inhibit the Viral RT or the Viral Protease

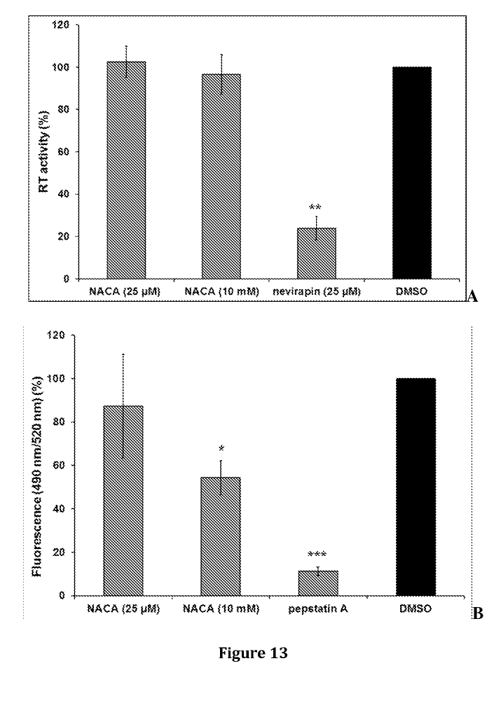

Most compounds that show anti-HIV activity and which have been developed into anti-HIV drugs are RT or protease inhibitors. Therefore, NACA was examined for its ability to inhibit these two viral proteins using two commercially available kits. The results in FIG. 13 show that NACA has no effect on either enzyme at 25 μM, but a significant effect on the protease activity at 10 mM.

For the data shown in FIG. 13A, a poly-A plate was incubated with the reaction. buffer and subsequently, the viral sample (1:500 dilution) together with the NACA, nevirapin (inhibition control) or DMSO (vehicle control) was added and incubated overnight. The plate was exposed to the tracer and the substrate was added. The plate was incubated in the dark and read at 405 nm. The value for the vector control (DMSO) was set to 100% in each individual repeat and all other values were normalized to this reference value. The data shown in FIG. 13A are means of three independent experiments; the error bars represent the SEM. P-values are indicated if significant (*: p <0.05; **: p <0.01; ***: p <0.001).

For the data shown in FIG. 13B, the HIV-1 protease together with NACA, a positive control (pepstatinA) or DMSO (vehicle control) were added to a dark-bottom 96 well plate. The plate as was incubated and protease substrate was added to all wells. The fluorescence was measured at Ex/Em=490 nm/520 nm. The value for the vector control (DMSO) was set to 100% in each individual repeat and all other values were normalized to this reference value. The data are means of three independent experiments; the error bars represent the SEM. P-values are indicated if significant (*: p <0.05; **: p <0.01; ***: p <001).

Hence, the inhibition of HIV replication by NACA is not due to the inhibition of the RT, but could be due to an effect on the viral protease.

2.3. Effect on Viral integration

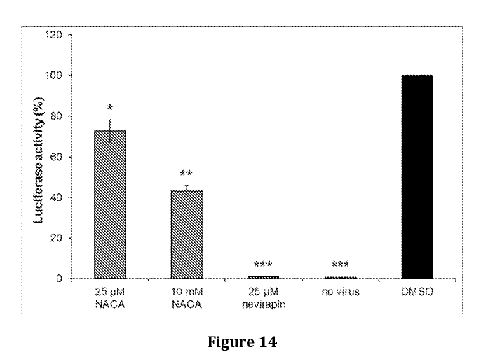

To determine additional targets of NACA inhibition within early stage processes of viral integration (viral entry to integration into the host genome), an infectivity assay was performed. TZM-b1 cells contain a luciferase gene that is under control of the HIV-1 promoter. Upon viral entry, transcription and integration into the host genome, luciferase is expressed and can convert the added substrate to the fluorescent product. The amount of measured product is quantitatively correlated to the amount of integrated virus in the teat culture. The results show (FIG. 14) that NACA has already a significant effect on viral integration at lower concentrations (25 μM), but a more predominant effect at 10 mM.

For the data in FIG. 14, TZM-b1 cells were infected with a diluted virus suspension in a total of 0.2 ml DMEM medium in the presence of the 25 μM or 10 mM NACA, an inhibition control (nevirapin) or a vehicle control (DMSO). Additionally, one well was treated with medium only instead of the virus dilution (no virus, negative control). After 40 h, the cells were assayed for luciferase activity. The value for the vehicle control was set to 100% in each individual repeat and the negative control was set to 0%. All other values were normalised these reference values. The data are means of three independent experiments; the error bars represent the SEM. P-values were calculated between the vehicle control and the individual treatments, P-values are indicated if significant (*: p <0.05; **: p <0.01; ***: p <0.001).

This indicates activity of the compound at a stage before or including integration as well as that the process is dose-dependent.

7.4. Production of Viral Particles

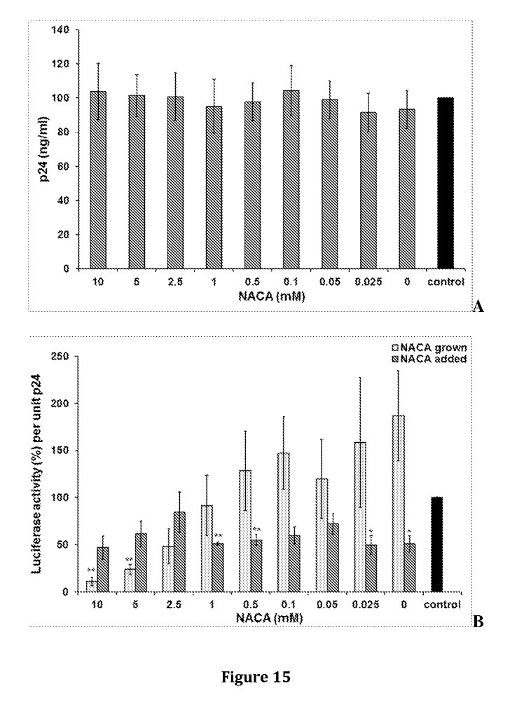

The production of viral particles in the presence of the compounds and their infectivity was investigated to give a complete picture of the effects of NACA on the virus and its life cycle. The amount of p24 (FIG. 15A) was not affected by any of the addition of NACA and it can therefore be assumed that the amount of produced virus is stable. However, there was a dose-dependent effect on the infectivity of the particles produced in the presence of NACA. At low concentrations (0 mM to 1 mM) NACA had an auxiliary effect on the effectivity of the HIV particles produced in the presence of NACA (NACA grown), while at higher concentrations (5 mM to 10 mM) the effect was reversed and the infectivity of the particles decreased significantly. However, this effect was not visible when NACA was added after the production of the viral particles (NACA added).

For the data in FIG. 15A, ACH-2 cells were cultured with 100 nM PMA in the presence of the indicated amount of NACA, DMSO (vector control=0 mM NACA) or no additive (control). After 3 days, the supernatant was clarified and the amount of p24 was measured by ELISA. The value for the control was set to 100% in each individual repeat and all other values were normalized these reference values. The data are means of three independent experiments; the error bars represent the SEM. P-values were calculated between the vehicle control and the individual treatments. P-values are indicated if significant (*: p<0.05; **: p <0.01; ***: p <0.001).

For the data in FIG. 15B, the clarified supernatant was further used for an infectivity test without the addition of any compound to the wells. The NACA amount stated in the graph refers to the amount in which the HIV producing cells were grown. TZM-b1 cells were infected with the diluted virus suspensions in a total of 0.2 ml DMEM (total dilution of virus supernatant: 1:100). After 40 h, the cells were assayed for luciferase activity. Additionally, a control experiment was performed, in which virus particles produced in the control set up, were used for the infectivity test. The same amount of NACA that could have been carried over with the virus containing supernatant in the original experiment was added to the infectivity test (final concentration: 1/100 of the indicated amount in the viral growth culture). The value for the control was set to 100% in each individual repeat and all other values were normalised these reference values. The data are means of three independent experiments; the error bars represent the SEM. P-values were calculated between the vehicle control and the individual treatments. P-values are indicated if significant (*: p <0.05; **: p <0.01; ***: p <0.001).

What is claimed is:

1. A method of reducing human immunodeficiency virus (HIV) integration in a mammalian cell comprising administering to the cell a compound comprising an effective amount Of NAGA.

2. The method of claim 1, wherein viral integration of HIV is decreased by at least 50%.

3. The method of claim 1, wherein viral integration of HIV is decreased by at least 90%.

4. The method of claim 1, wherein the mammalian cells are human cells.
